(19) United States
(12) Patent Application Publication
(10) Pub. No.: US 2024/0060955 A1
(43) Pub. Date: Feb. 22, 2024

(54) SIMULTANEOUS AND SELECTIVE WASHING AND DETECTION IN ION SELECTIVE ELECTRODE ANALYZERS

(71) Applicant: BECKMAN COULTER, INC., Brea, CA (US)

(72) Inventors: Yuko NAKASHIMA, Tokyo (JP); Kumiko YAMADA, Tokyo (JP)

(21) Appl. No.: 18/269,828

(22) PCT Filed: Nov. 17, 2021
(86) PCT No.: PCT/US2021/059610

§ 371 Date: Jun. 27, 2023

Related U.S. Application Data
(60) Provisional application No. 63/132,022, filed on Dec. 30, 2020.

(51) Int. Cl. G01N 33/487; G01N 27/333

(52) U.S. Cl. CPC G01N 33/48707; G01N 27/333

(57) ABSTRACT

A considerable amount of time is required for calibration and compliance service for electrolyte measuring devices with ion selective electrode analyzers in most clinical or diagnostic laboratory settings. Often a user has to make trade-offs between improving diagnostic accuracy and processing higher workloads faster and more predictably. Current electrolyte measuring devices with ion selective electrodes are unable to balance the increased requirements for accuracy and speed. The presently claimed and described technology provides an improved device for an ion selective electrode analyzer. The presently claimed and described technology also provides methods for simultaneous and selective washing of components of the ion selective electrode analyzer and methods for simultaneous and selective analysis of samples using the ion selective electrode analyzer in an automated chemical analyzer.

### CROSS-REFERENCE TO RELATED APPLICATIONS

The present application claims priority to U.S. Provisional Application No. 63/132,022, filed Dec. 30, 2020, which is incorporated by reference herein in its entirety.

### FIELD

Various aspects of the present disclosure relate to simultaneous and selective washing of ion selective electrode analyzer components and simultaneous and selective electrolyte detection using an ion selective electrode analyzer. Additional aspects include an improved ion selective electrode analyzer device.

### BACKGROUND

Automated chemical analyzers are commonly used in clinical chemistry sampling and analyzing applications. Automated analytical equipment, such as automated analytical chemistry workstations, can efficiently perform clinical analysis on a large number of samples, with tests being run concurrently or within short time intervals. Efficiencies result in part because of the use of automated sample identification and tracking. This equipment can automatically prepare appropriate volume samples and can automatically set the test conditions needed to perform the scheduled tests. Test conditions can be independently established and tracked for different testing protocols simultaneously in progress within a single test station, facilitating the simultaneous execution of several different tests based on different chemistries and requiring different reaction conditions. Automated analytical equipment is particularly well-suited for high volume testing environments, such as those existing in many hospitals and in centralized testing laboratories, because the automatic sample handling allows for more precise sample identification and sample tracking. Automatic handling and tracking of samples significantly reduces the opportunity for human error or accidents that can lead to either erroneous test results or undesirable contamination.

An ion selective electrode is an important part of an automated chemical analyzer. When measuring the concentration of electrolytes (ions such as Sodium, Potassium, and Chloride) in a sample with an automatic chemical analyzer, an ion selective electrode (ISE) is used as a typical method. In the electrolyte measuring device with ISE, a user desires high throughput, reproducibility, and minimum maintenance. In most clinical or diagnostic laboratory settings, a considerable amount of time is required for calibration and compliance service for electrolyte measuring devices with ISE. Often a user has to make trade-offs between improving diagnostic accuracy and processing higher workloads faster and more predictably. Current electrolyte measuring devices with ISE are unable to balance the increased requirements for accuracy and speed.

In general ISE methods, an internal standard solution (a solution of known concentration) is measured for each sample measurement. The sample's electrolyte concentration is calculated from the voltage difference between the internal standard solution and sample diluent. It is desired to keep the test solution inside the electrode for a long time. If the test solution's residual time is short, the measurement performance will deteriorate if an electrode with deteriorated responsiveness is used.

In some ISE methods, the measurement operation is performed in the order of the voltage measurement of the internal standard solution, the voltage measurement of the sample diluent, and the washing operation with the internal standard solution in one cycle. With this method, it is necessary to shorten the test liquid retention time inside the electrode to increase the throughput, and it is an issue to achieve both throughput and data reproducibility.

In addition, if a measurement is not performed continuously, the initial voltage value becomes unstable due to the electrode film's drying. Therefore, it is necessary to perform an electrode conditioning operation (e.g., to flow the internal standard solution into the electrode) before measurement depending on the measurement interval.

However, if the conditioning operation is performed before the measurement, the start of electrolyte measurement will be delayed, and the processing capacity of the entire ISE will decrease depending on the measurement conditions, such as the number of ordering photometric items.

In the prior art described in JP 3610111, the above problems can be solved. In the prior art, an ion selective analyzer has a bypass line for aspirating the test liquid in the dilution pot without passing through the electrodes. In this method, the reagents used include an internal standard solution and a sample diluent and are always measured alternatively without a washing operation.

With this measurement method, the sample diluent and the internal standard solution can be dispensed and stirred during the resting potential measurement, so it is possible to secure a long period of time to measure the potential within one cycle. Further, the electrodes are always filled with the sample diluent or the internal standard solution, so no conditioning operation is required even with long intervals of measurement. And the measurement can start at any time. Therefore, it is possible to provide an analyzer with high processing capacity regardless of sample measurement conditions, such as the number of photometric items.

However, this method has some problems. Imprecise data may be obtained when measuring high/low concentration samples. Since the previous sample's residual liquid contaminates the internal standard solution, a sample's data reproducibility, especially when there are large variations in concentration, may be flawed.

Another problem is the increased user maintenance time. Since the remaining components of the sample diluent almost always remain in the dilution pot and/or tubing, dirt easily accumulates in the dilution pot and/or tubing, which may cause clogging. Dirt and/or clogging may cause calibration errors and abnormal measurement values. To prevent this, the user needs to perform frequent and many maintenances (cleaning, replacement of parts, etc.).

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

The inventors have recognized the need for an improved ion selective electrode device, as well as methods for the simultaneous and selective washing of ion selective electrode components and simultaneous and selective detection of electrolytes with an ion selective electrode analyzer.

One aspect of the presently disclosed and claimed technology includes an ion selective electrode analyzer, the ion selective electrode analyzer comprising: at least one reagent supply line, the at least one reagent supply line further comprising at least one reagent, a dilution pot for receiving the reagent; a flow cell downstream from the dilution pot; a flow cell line operatively connected with the flow cell; a bypass line operatively connected with the dilution pot; a drain line; a flushing liquid line; at least three pumps; a first valve, wherein the first valve is a three-way valve or a pinch valve, a second valve, wherein the second valve is a three-way valve, and a third valve, wherein the third valve is a two-way valve.

One aspect of the presently disclosed and claimed technology includes methods of analyzing a biological sample with an ion selective electrode analyzer, a method of analyzing a biological sample with an ion selective electrode analyzer, the method comprising the steps of: providing the ion selective electrode analyzer, the ion selective electrode analyzer comprising: at least one reagent supply line, the at least one reagent supply line further comprising at least one reagent, a dilution pot, a flow cell, optionally, a flow cell line, optionally, a bypass line, optionally, a drain line, and optionally, at least two pumps, a first pump and a second pump; mixing a volume of a biological sample and a volume of the reagent in the dilution pot to produce a diluted biological sample; aspirating the diluted biological sample from the dilution pot into the flow cell for analysis; simultaneously analyzing the diluted biological sample in the flow cell while dispensing the reagent from the reagent supply line into the dilution pot wherein the reagent is used for a bypass line wash, and then dispensing the reagent from the reagent supply line into the dilution pot, wherein the reagent is used for a flow cell line wash.

In some aspects, the reagent is aspirated into the bypass line for a bypass line wash prior to washing the bypass line. The bypass line wash may include, but is not limited to, washing the dilution pot, washing the bypass line, and washing the drain line. In some aspects, the reagent is aspirated to the flow cell line for a flow cell line wash after the diluted biological sample is analyzed. The flow cell line wash may include, but is not limited to, washing the dilution pot, washing the flow cell, washing the flow cell line, and washing the drain line. In some aspects, the diluted biological sample and/or the reagent are aspirated into the bypass line before and/or after aspirating into the flow cell line. In other aspects, wherein after washing the flow cell line, the method further comprises the step of dispensing the reagent from the reagent supply line into the dilution pot, and aspirating the reagent to the flow cell line, and calibrating the flow cell using the reagent.

In another aspect of the method, the first pump is configured to pump the reagent from the reagent supply line, and the second pump is configured to aspirate the reagent and/or the biological sample. The reagent may comprise an internal standard.

In another aspect of the method, the at least one reagent supply line is configured to dispense at least a first reagent and at least a second reagent. Alternatively, the ion selective electrode analyzer further comprises a second reagent supply line, wherein the first reagent supply line comprises a first reagent and the second reagent supply line comprises a second reagent. In a further aspect, the first reagent comprises an internal standard, wherein the internal standard is used to wash the flow cell line and/or calibrate the flow cell, and the second reagent comprises a buffer, wherein the buffer is used to dilute the biological sample and/or to wash the bypass line.

In one aspect of the method, the ion selective electrode analyzer further comprises at least a first valve, wherein the first valve is a pinch valve, with a Y-shape connector, or a three-way valve. In another aspect, the ion selective electrode analyzer further comprises a second valve, wherein the second valve is a three-way valve.

In one aspect of the method, the ion selective electrode analyzer further comprises at least a third pump. In another aspect, the ion selective electrode analyzer further comprises a flushing liquid line and a third valve, wherein the third valve is a two-way valve.

In one aspect of the method, the ion selective electrode analyzer further comprises a fourth pump. In another aspect, the fourth pump is configured to pump a second reagent from the second reagent supply line.

One aspect of the presently disclosed and claimed technology includes an ion selective electrode analyzer comprising: at least one reagent supply line, the at least one reagent supply line further comprising at least one reagent, a dilution pot; a flow cell; a flow cell line; a bypass line; optionally, a drain line; and optionally, at least two pumps, a first pump, and a second pump; wherein the ion selective electrode analyzer is configured to analyze at least one hundred biological samples per hour, and is further configured to calibrate the flow cell after each biological sample is analyzed.

One aspect of the presently disclosed and claimed technology includes an ion selective electrode analyzer comprising: at least one reagent supply line, the at least one reagent supply line further comprising at least one reagent, a dilution pot; a flow cell; flow cell line; optionally, a bypass line; optionally, a drain line, and optionally, at least two pumps, a first pump, and a second pump; wherein the ion selective electrode analyzer is configured to continuously wet a flow cell line or a flow cell by alternating between aspirating the diluted biological sample from the dilution pot and the reagent from the one reagent supply line into the flow cell and/or flow cell line.

One aspect of the presently disclosed and claimed technology includes a method for continuous wetting of a flow cell line or a flow cell comprising the steps of providing an ion selective electrode analyzer, the ion selective electrode analyzer comprising at least one reagent supply line, the at least one reagent supply line further comprising at least one reagent, a dilution pot; a flow cell; flow cell line; optionally, a bypass line; optionally, a drain line, and optionally, at least two pumps, a first pump, and a second pump; mixing a volume of a biological sample and a volume of the reagent in the dilution pot to produce a diluted biological sample, and alternating between aspirating the diluted biological sample from the dilution pot and the reagent from the one reagent supply line into the flow cell and/or flow cell line.

These and other advantages, aspects, and novel features of the present disclosure, as well as details of an illustrated embodiment thereof, will be more fully understood from the following description and drawings.

### BRIEF DESCRIPTION OF THE FIGURES

Embodiments of the present disclosure will now be described, by way of example only, with reference to the attached Figures, wherein:

FIG. 1 illustrates one embodiment of the disclosure where an ion selective electrode analyzer comprises two (2) three-way valves.

FIG. 2 illustrates an embodiment of the disclosure where an ion selective electrode analyzer comprises a pinch valve and a Y-shape connector.

FIG. 3 illustrates a flushing sequence according to an embodiment of the disclosure.

FIG. 4 illustrates a washing sequence according to an embodiment of the disclosure.

FIG. 5 illustrates a first measurement flow according to an embodiment of the disclosure.

FIG. 6 illustrates a second measurement flow according to an embodiment of the disclosure.

FIG. 7 is a configuration diagram explaining a schematic configuration of an automated chemical analyzer comprising an ion selective electrode analyzer.

FIG. 8 illustrates a comparison of the method cycle according to one embodiment of this disclosure versus a conventional method cycle.

FIG. 9 shows the carry-over rate (%) from a urine sample (high-concentration sample) to a serum sample measured in a conventional system compared to an embodiment of the disclosure.

FIG. 10 shows the coefficient of variation (CV %) of standard-concentration sample and high-concentration sample measured in a conventional system compared to an embodiment of the disclosure.

### DETAILED DESCRIPTION

Various embodiments will be described in detail with reference to the drawings, wherein like reference numerals represent like parts and assemblies throughout the several views. It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

As used herein and in the appended claims, the singular forms “a,” “an,” and “the” include plural reference unless the context clearly indicates otherwise.

Unless defined otherwise, all technical and scientific terms used herein have the same meaning as commonly understood to one of ordinary skill in the art to which this disclosure belongs.

As described herein, an improved ion selective electrode analyzer device, as well as methods for the simultaneous and selective washing of ion selective electrode analyzer components and simultaneous and selective detection of electrolytes using an ion selective electrode analyzer are disclosed.

Quantitation of conventional chemistry analytes is typically based on one of two measurements: (1) Measurement of light (photometry or spectrophotometry) or (2) Measurement of electrochemical potential (potentiometry). While the examples below discuss potentiometry (the measurement of electrochemical potential), other analysis methods, such as photometry or spectrophotometry, may be utilized. Potentiometry, using an ion selective electrode analyzer, is an analytical technique used to determine the activity of ions in an aqueous solution by measuring the electrical potential. An ion selective electrode analyzer may be used to simultaneously analyze for sample electrolytes, including, but not limited to, sodium, potassium, calcium, chlorine, and carbon dioxide.

An ion selective electrode analyzer for Na⁺, K⁺, and Cl⁻ employs crown ether membrane electrodes for sodium and potassium and a molecular oriented PVC membrane for chloride specific for each ion of interest in the sample. An electrical potential is developed according to the Nernst Equation for a specific ion. When compared to an internal reference solution, this electrical potential is translated into voltage and then into the sample's ion concentration—Tietz, N. W., editor, Fundamentals of Clinical Chemistry, 3rd Edition, W. B. Saunders 1987.

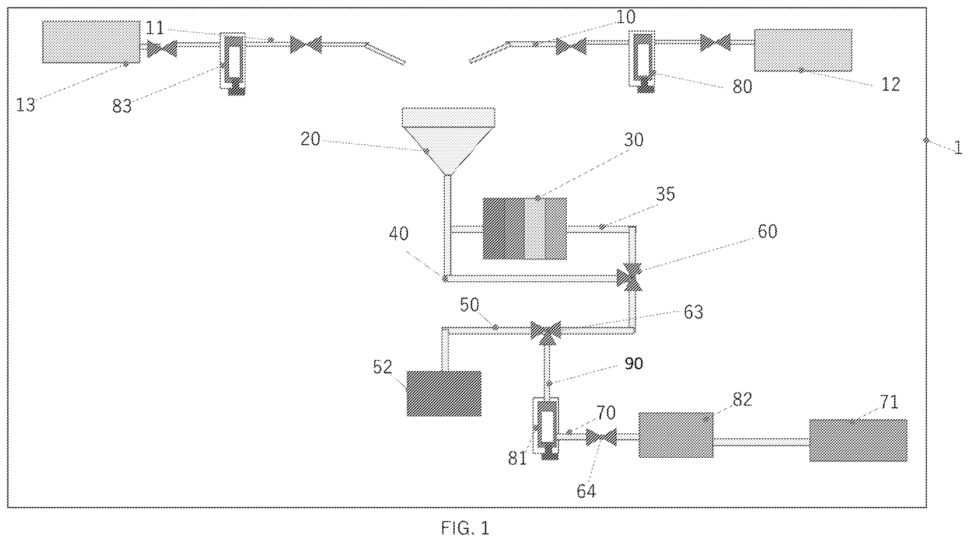

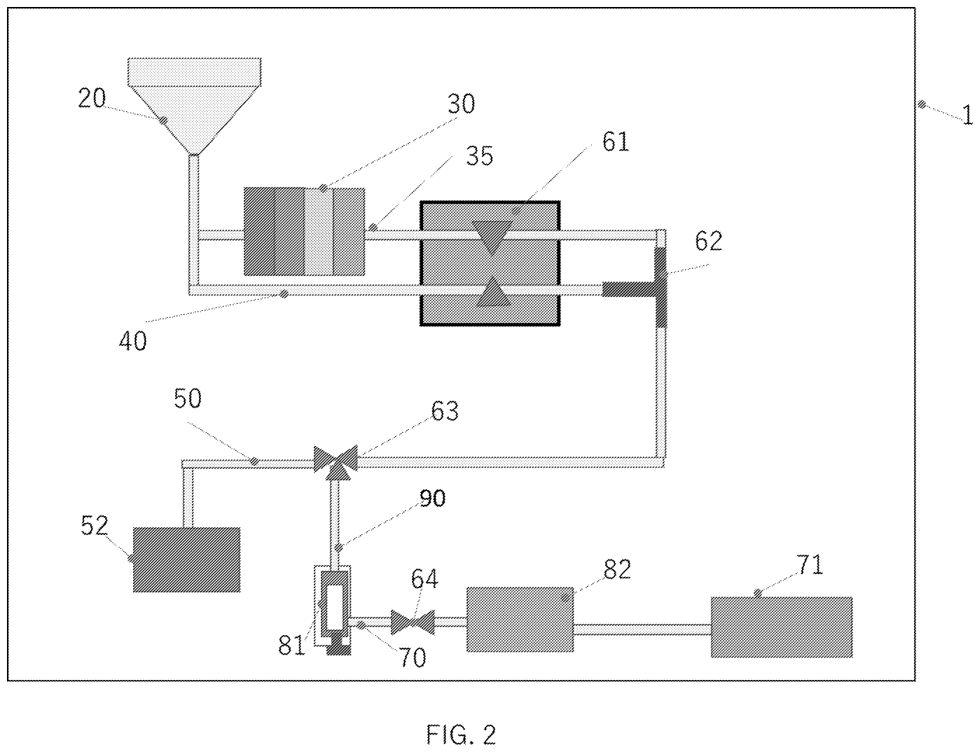

According to at least one embodiment of the presently described and claimed technology, an ion selective electrode analyzer configuration is described below with reference to FIGS. 1 and 2. FIG. 1 shows an ion selective electrode analyzer according to an embodiment of the disclosure. The ion selective electrode analyzer 1 comprises at least one reagent supply line 10, the at least one reagent supply line 10 further comprising at least one reagent, a dilution pot 20 for receiving the reagent, a flow cell 30 downstream from the dilution pot 20, a flow cell line 35 operatively connected with the flow cell 30, a bypass line 40 operatively connected with the dilution pot 20, a drain line 50, a flushing liquid line 70, at least three pumps 80,81,82, a first valve, wherein the first valve is a three-way valve 60, a second valve, wherein the second valve is a three-way valve 63, and a third valve, wherein the third valve is a two-way valve 64. In some embodiments, the ion selective electrode analyzer may further comprise a drain 52 operatively connected to the drain line 50.

The reagent supply line 10 may be configured to dispense at least one reagent, alternatively at least two reagents, alternatively a plurality of reagents. The reagents may be housed in a reagent container 12. The reagent container 12 may contain one or more compartments for retaining one or more different reagents.

In some embodiments, the ion selective electrode analyzer may further comprise a second reagent supply line 11 comprising at least one reagent. In this embodiment, the second reagent supply line 11 has a reagent container 13 separate from the reagent container 12 of the first reagent supply line 10. In this embodiment, the ion selective electrode analyzer further comprises a fourth pump 83 that is connected to the second reagent supply line 11.

In FIG. 1, the first valve is a three-way valve 60. This three-way valve 60 is used to select the flow cell line 35 or bypass line 40 for aspirating. In FIG. 2, the first valve is a pinch valve 61. When the ion selective electrode analyzer 1 comprises a pinch valve 61, it further comprises a Y-shape connector 62. The pinch valve 61 pinches the flow cell line 35 or bypass line 40. The Y-shape connector 62 operatively connects the flow cell line 35, bypass line 40, and drain line 50.

To dispense or aspirate a fixed or variable volume, the ion selective electrode analyzer 1 comprises at least one pressure altering mechanism. This pressure altering mechanism may be a pump, such as a peristaltic pump, roller pump, or syringe pump. As illustrated in FIGS. 1 and 2, at least one of the first pump 80 or second pump 81 may be a syringe pump. In some embodiments, both the first pump 80 and the second pump 81 are syringe pumps. One advantage of a syringe pump is the ability to dispense or aspirate precise amounts to minimize the consumption of, for example, a reagent.

In FIGS. 1 and 2, the second pump 81 is connected to the drain line 50 via the second valve 63, wherein the second valve 63 is positioned at or near the middle of the drain line 50. The position of the second valve 63 separates the drain line 50 into a pre-flushing portion and a post-flushing portion. In FIGS. 1 and 2, the third valve 64 is positioned at or near the middle of the flushing liquid line 70, the flushing liquid line 70 connecting the second pump 81 and the third pump 82. The third pump may be operatively connected to a source of flushing liquid 71, and the third pump 82 may be configured to pump a flushing liquid. The flushing liquid can be any liquid suitable for flushing lines of an ion selective electrode analyzer. A non-limiting example of a suitable flushing liquid includes deionized water.

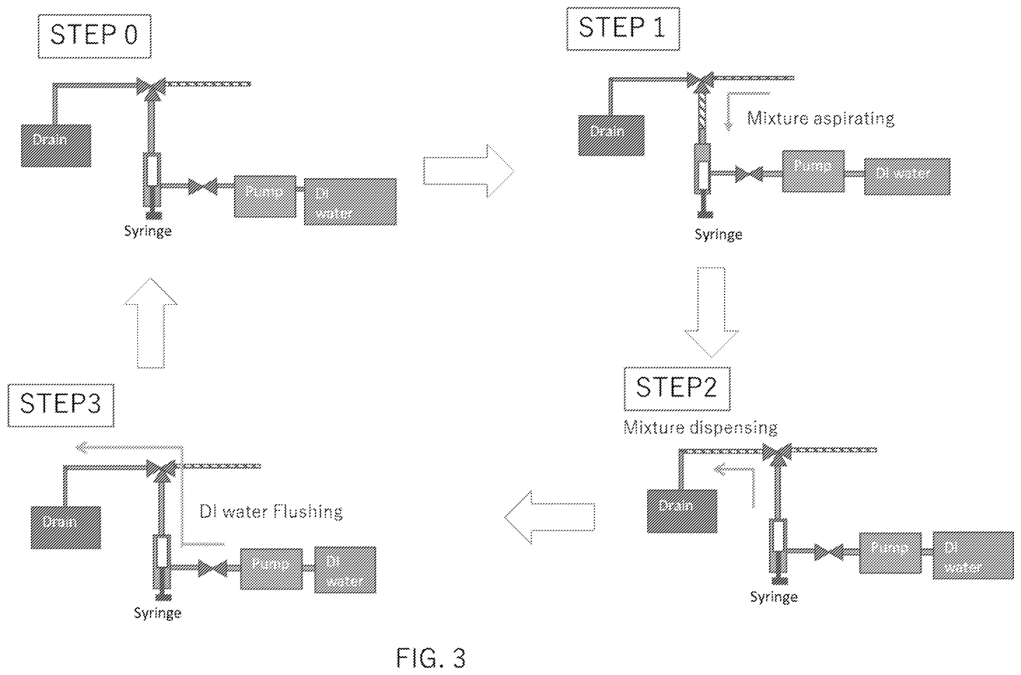

In an ion selective electrode analyzer according to an embodiment of the disclosure, the tube 90 capacity between the second valve 63 and the second pump 81 is greater than the volume the second pump 81 is configured to aspirate. As illustrated in FIG. 3, the increased volume ensures that the aspirated mixture does not enter the syringe pump. The flushing step further prevents mixture diffusion in the syringe pump. This flush operation and the large capacity of the tube between syringe and valve can reduce issues that may arise due to accumulation of residual sample on the aspirating syringe and can significantly reduce maintenance (washing/replacement) required aspirating syringe. In addition, the piping shown in FIG. 1 eliminates the need for consumable piping (e.g., roller pump tubing, pinch valve piping) and reduces the time and effort required to replace the piping and/or tubing.

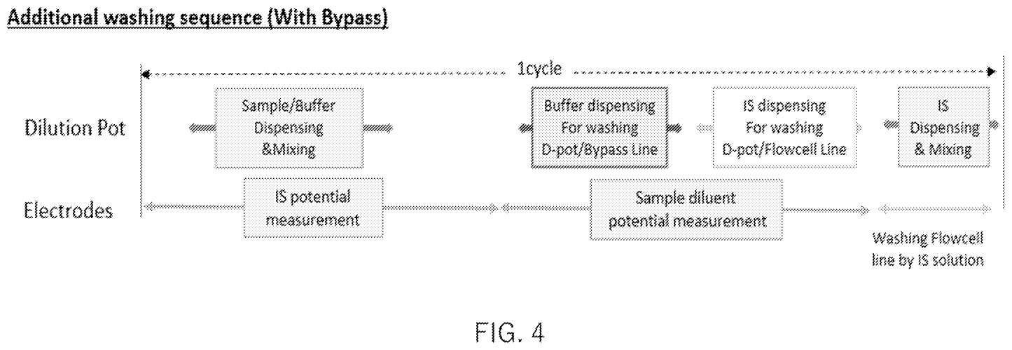

According to at least one embodiment of the presently described and claimed technology, methods of using an ion selective electrode analyzer are described below with reference to FIGS. 4 to 6.

In an ion selective electrode analyzer 1 with a bypass line 40, at least two (2) washing operations may be added to one (1) measurement cycle. In this system, by effectively using the bypass line 40, the at least two (2) washing operations can be added without increasing the measurement cycle time while maintaining the test solution's current retention time in the flow cell line. Additionally, the addition of at least two washing operations reduces the carry-over between reagents, and the data reproducibility of the high/low concentration sample is improved. Also, the amount of residual sample in the dilution pot/tubing after the measurement is reduced, resulting in a reduction of the maintenance (replacement or cleaning) time of these parts. See FIG. 4.

In one embodiment of the disclosure, the carry-over is reduced by about 25% compared to conventional ISE methods. In this embodiment, the cleaning maintenance only has to be done, for example, about once or month or about every 30,000 tests. This contrasts with the conventional ISE methods, wherein cleaning maintenance is done about weekly or about every 7,500 tests. Additionally, in this embodiment, the ion selective electrode analyzer parts last for at least seven (7) years, and in some embodiments, these parts require no replacement. This is in contrast to the conventional method, where parts are replaced monthly. This low-frequency maintenance is one of the unexpected results of the improved ion selective electrode analyzer device and related methods.

One embodiment of the disclosure is a method of analyzing a biological sample with an ion selective electrode analyzer 1, the method comprising the step of providing the ion selective electrode analyzer 1, the ion selective electrode analyzer 1 comprising: at least one reagent supply line 10, the at least one reagent supply line 10 further comprising at least one reagent, a dilution pot 20, a flow cell 30, a flow cell line 35, a bypass line 40, a drain line 50, and at least two pumps, a first pump 80, and a second pump 81; mixing a volume of a biological sample and a volume of the reagent in the dilution pot 20 to produce a diluted biological sample; aspirating the diluted biological sample from the dilution pot 20 into the flow cell 30 for analysis; simultaneously analyzing the diluted biological sample in the flow cell 30 while dispensing the reagent from the reagent supply line 10 into the dilution pot 20, and aspirating the reagent to the bypass line 40, wherein the reagent is used for a bypass line wash to wash the bypass line, and then dispensing the reagent from the reagent supply line 10 into the dilution pot 20 and aspirating the reagent to the flow cell line 35 after the diluted biological sample is analyzed, wherein the reagent is used for a flow cell line wash to wash the flow cell line. In some embodiments, the diluted biological sample is aspirated into the bypass line 40 before aspirating into the flow cell 30. In some embodiments, wherein after washing the flow cell line 35, the method further comprises the step of dispensing the reagent from the reagent supply line 10 into the dilution pot 20, and aspirating the reagent to the flow cell line 35, and calibrating the flow cell 30 using the reagent. In this embodiment, the reagent is an internal standard.

In one embodiment of the disclosure, these method steps can be performed simultaneously, sequentially and/or continually.

FIG. 4 illustrates a measurement cycle with two (2) additional washings. In this method, reagent supply line 10 is configured to dispense at least a first reagent and at least a second reagent. Alternatively, the ion selective electrode analyzer 1 may comprise a second reagent supply line 11, wherein the first reagent supply line 10 comprises a first reagent and the second reagent supply line 11 comprises a second reagent. In this two-fluid system embodiment, the first reagent comprises an internal standard, wherein the internal standard is used to wash the flow cell line 35 and/or calibrate the flow cell 30, and the second reagent comprises a buffer. Any suitable buffer may be used, and non-limiting examples of a buffer include phosphate-buffered saline, neutral salts, deionized water, or mixtures thereof. In this embodiment, the buffer is used to dilute the biological sample and/or to wash the bypass line 40.

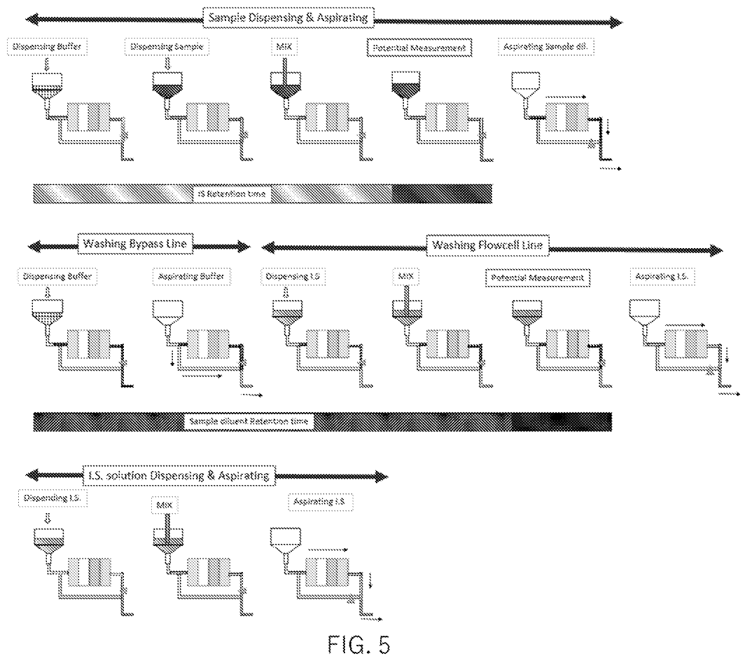

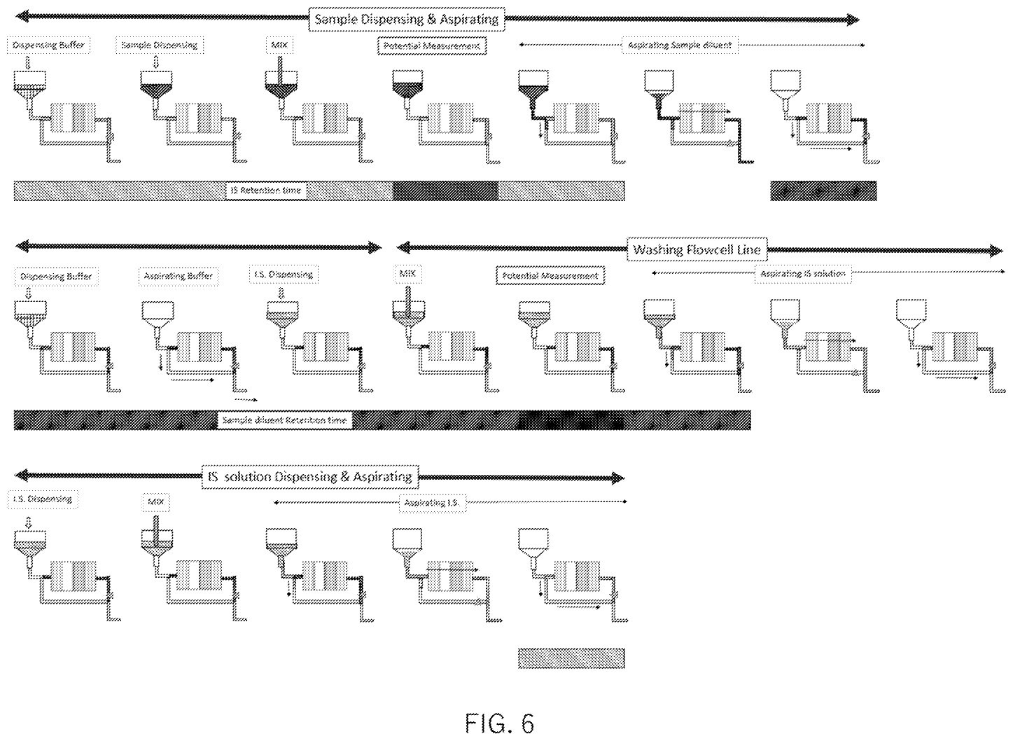

Measurement flows according to at least two embodiments of the disclosure are illustrated in FIGS. 5 and 6. In FIG. 5, the measurement flow is simplified, and no bypass suction of the diluted sample or reagent is required when replacing the test solution in the flow cell (e.g., electrode).

The biological sample may be from a mammal, preferably a human, and includes, but is not limited to blood, plasma, serum, saliva, urine, cerebrospinal fluid, lacrimal fluid, perspiration, gastrointestinal fluid, amniotic fluid, mucosal fluid, pleural fluid, and sebaceous oil. Prior to analysis, the biological sample is mixed with the reagent, which may include, but is not limited to, an internal standard, a buffer, or a sample diluent. The volume of biological sample mixed with the reagent is at least about 5 μL, alternatively at least about 10 μL, alternatively at least about 15 μL, alternatively at least about 20 μL. The volume of reagent mixed with the biological sample is at least about 300 μL, alternatively at least about 350 μL, alternatively at least about 400 μL, alternatively at least about 450 μL, alternatively at least about 500 μL, alternatively at least about 550 μL, alternatively at least about 600 μL, alternatively at least about 650 μL.

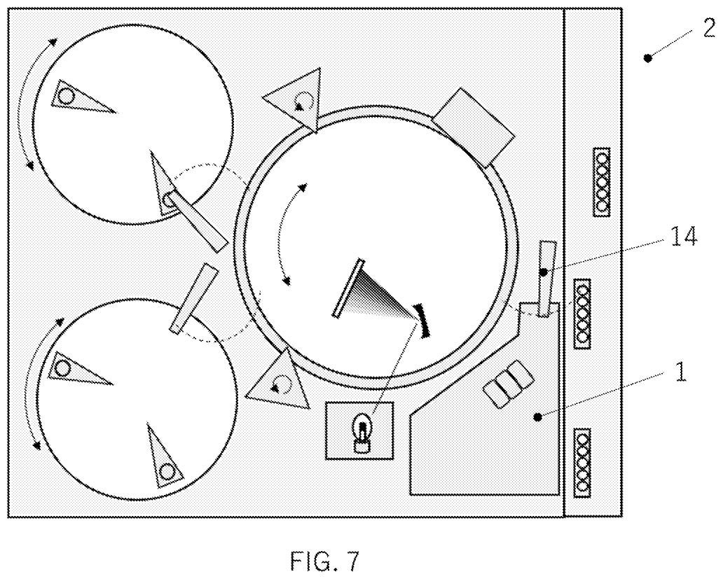

FIG. 7 illustrates an embodiment of the disclosure where an ion selective electrode analyzer 1 is incorporated into an automated chemical analyzer 2. The automated chemical analyzer 2 comprises a sample station 14 configured to dispense the biological sample.

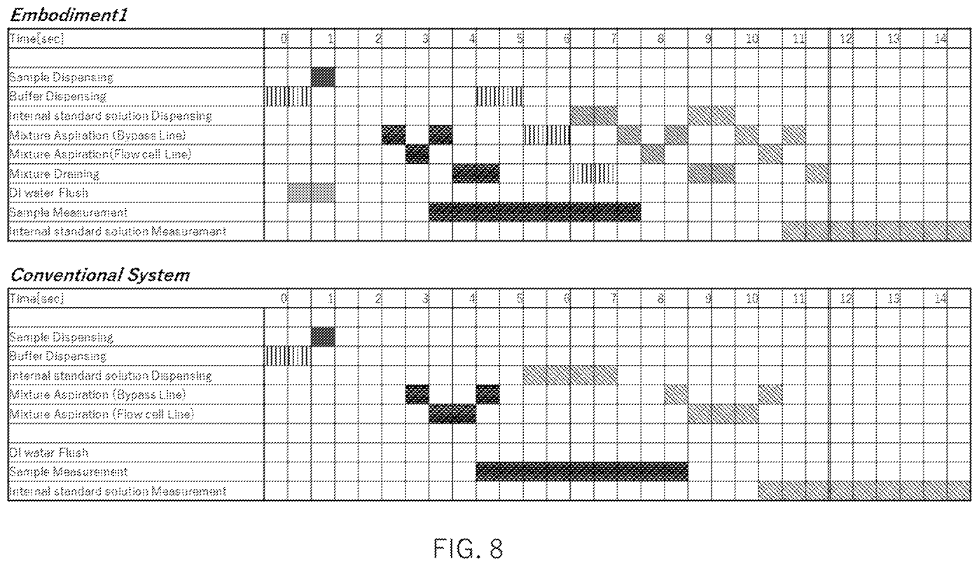

Even with additional washings, the measurement cycle is still relatively short. In some embodiments, the cycle is about 20 seconds, alternatively about 15 seconds, alternatively about 12 seconds, alternatively about 10 seconds. As illustrated in FIG. 8, the measurement cycle is about 12 seconds. This short cycle time reduces the need for frequent cleaning maintenance and/or frequent parts replacement. FIG. 8 illustrates a comparison of the method cycle according to one embodiment of this disclosure (Embodiment 1) versus a conventional method cycle.

Additional examples are provided below.

### Examples

### Example 1: Carry-Over Value (Urine Sample Serum Sample)

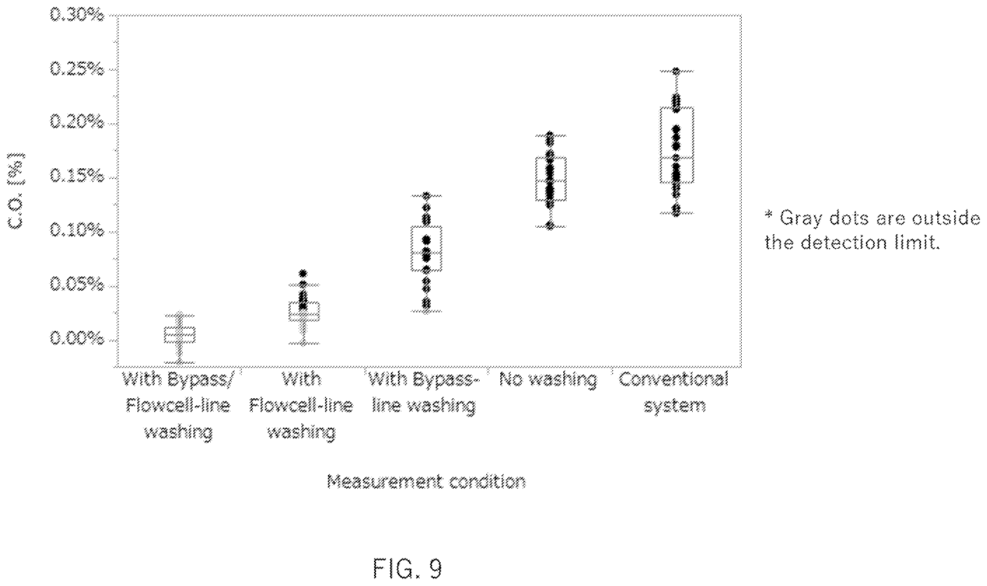

The graph of FIG. 9 shows the carry-over rate (%) from a urine sample (high-concentration sample) to a serum sample using a conventional system with a bypass line and using an ion selective electrode analyzer of this disclosure (“new system”).

Carry-over in the double wash addition sequence (with bypass/flowcell washing) was less than about ¼ of the conventional sequence's carry-over. No washing was done in the conventional system.

When comparing the new system with the conventional system, while the accumulation of dirt on dilution pots and tubing was reduced to the same extent, it is expected that the maintenance frequency/time of the dilution pots and tubing in the new system will be reduced to about ¼ of the maintenance frequency/time of the conventional system.

### Example 2: Reproducibility

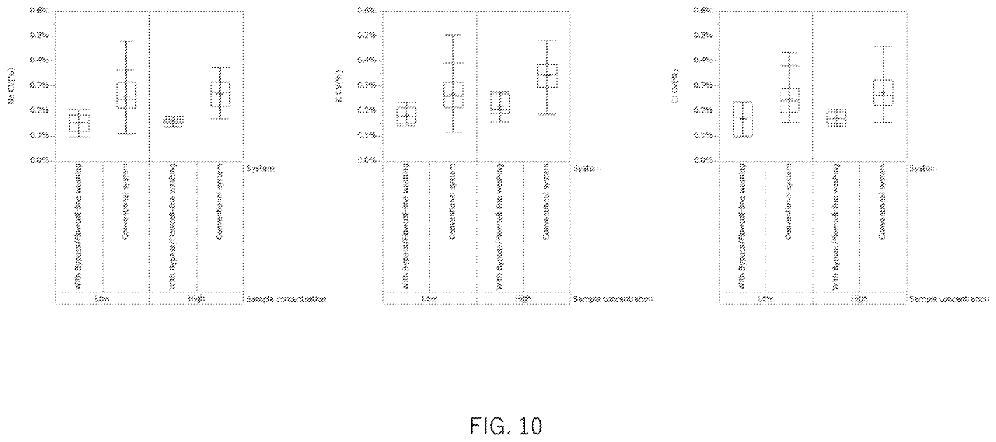

FIG. 10 shows that the data reproducibility CV % (N=20×8 rounds) of low-concentration sample and high-concentration sample measured in a conventional system and measured in the new system with flow cell/bypass line washing. The data reproducibility was better in the new system as compared to the conventional system.

The above description is illustrative and is not restrictive. Many variations of the invention will become apparent to those skilled in the art upon review of the disclosure. Therefore, the scope of the invention should be determined not with reference to the above description, but instead should be determined with reference to the pending claims along with their full scope or equivalents.

All patents, patent applications, publications, and descriptions mentioned above are herein incorporated by reference in their entirety.

1. A method of analyzing a biological sample with an ion selective electrode analyzer (1), the method comprising the steps of:
providing the ion selective electrode analyzer (1), the ion selective electrode analyzer (1) comprising:
at least one reagent supply line (10), the at least one reagent supply line (10) further comprising at least one reagent, wherein the reagent is aspirated into the bypass line (40) prior to washing the bypass line (40) or wherein the reagent is aspirated to the flow cell line (35) for the flow cell line wash after the diluted biological sample is analyzed
a dilution pot (20);
a flow cell (30);
a flow cell line (35);
a bypass line (40);
a drain line (50), and
at least two pumps, a first pump (80) and a second pump (81);
mixing a volume of a biological sample and a volume of the reagent in the dilution pot (20) to produce a diluted biological sample;
aspirating the diluted biological sample from the dilution pot (20) into the flow cell (30) for analysis;
simultaneously analyzing the diluted biological sample in the flow cell (30) while dispensing the reagent from the reagent supply line (10) into the dilution pot (20) wherein the reagent is used for a bypass line wash,
and then dispensing the reagent from the reagent supply line (10) into the dilution pot (20), wherein the reagent is used for a flow cell line wash.

2. (canceled)

3. The method of claim 1, wherein the bypass line wash includes washing the dilution pot (20), washing the bypass line (40), and/or washing the drain line (50).

4. (canceled)

5. (canceled)

6. The method of claim 1, wherein the diluted biological sample and/or the reagent are aspirated into the bypass line (40) before and/or after aspirating into the flow cell line (35) and wherein the biological sample is selected from the group consisting of blood, plasma, serum, saliva, urine, cerebrospinal fluid, lacrimal fluid, perspiration, gastrointestinal fluid, amniotic fluid, mucosal fluid, pleural fluid, and sebaceous oil.

7. The method of claim 1, wherein after washing the flow cell line (35), further comprising the step of dispensing the reagent from the reagent supply line (10) into the dilution pot (20), and aspirating the reagent to the flow cell line (35), and calibrating the flow cell (30) using the reagent.

8. The method of claim 1, wherein the first pump (80) is configured to pump the reagent from the reagent supply line (10), and the second pump (81) is configured to aspirate the reagent and/or the biological sample.

9. (canceled)

10. The method of claim 1, wherein the reagent supply line (10) is configured to dispense at least a first reagent and at least a second reagent.

11. The method of claim 10, wherein the ion selective electrode analyzer (1) further comprises a second reagent supply line (11), wherein the first reagent supply line (10) comprises a first reagent and the second reagent supply line (11) comprises a second reagent.

12. The method of claim 10, wherein the first reagent comprises the internal standard, wherein the internal standard is used to wash the flow cell line (35) and/or calibrate the flow cell (30), and the second reagent comprises a buffer, wherein the buffer is used to dilute the biological sample and/or to wash the bypass line (40).

13. (canceled)

14. (canceled)

15. The method of claim 1, wherein the ion selective electrode analyzer (1) further comprises a Y-shape connector (62) and/or at least a first valve, wherein the first valve is a three-way valve (60) or a pinch valve (61).

16. (canceled)

17. The method of claim 15, wherein the first valve is used to select the flow cell line (35) or bypass line (40) for aspirating or dispensing the biological sample, or aspirating or dispensing the reagent.

18. The method of claim 15, wherein the ion selective electrode analyzer (1) further comprises a second valve, wherein the second valve is a three-way valve (63) and wherein at least one of the at least two pumps (80,81) is a syringe pump.

19. (canceled)

20. The method of claim 18, wherein at least one of the two pumps (80,81) is connected to the drain line (50) via the second valve (63), wherein the second valve (63) is positioned at or near the middle of the drain line (50).

21. The method of claim 20, wherein tube (90) capacity between the second valve (63) and the second pump (81) is greater than the volume the second pump (81) is configured to aspirate.

22. The method of claim 20, wherein the position of the second valve (63) separates the drain line (50) into a pre-flushing portion and a post-flushing portion.

23. The method of any one of claim 18, wherein the ion selective electrode analyzer (1) further comprises at least a third pump (82) and wherein the ion selective electrode analyzer (1) further comprises a flushing liquid line (70) and a third valve, wherein the third valve is a two-way valve (64).

24. (canceled)

25. The method of claim 23, wherein the flushing liquid line (70) connects the second pump (81) and the third pump (82), and the third valve (64) is positioned at or near the middle of the flushing liquid line (70).

26. (canceled)

27. (canceled)

28. The method of claim 23, wherein the ion selective electrode analyzer (1) further comprises a fourth pump (83), wherein the fourth pump (83) is connected to the second reagent supply line (11).

29-34. (canceled)

35. An ion selective electrode analyzer (1) comprising:
at least one reagent supply line (10), the at least one reagent supply line (10) further comprising at least one reagent,
a dilution pot (20) for receiving the reagent;
a flow cell (30) downstream from the dilution pot (20);
a flow cell line (35) operatively connected with the flow cell (30);
a bypass line (40) operatively connected with the dilution pot (20);
a drain line (50),
a flushing liquid line (70),
at least three pumps, a first pump (80), a second pump (81), and a third pump (82),
a first valve, wherein the first valve is a three-way valve (60) or a pinch valve (61),
a second valve, wherein the second valve is a three-way valve (63), and
a third valve, wherein the third valve is a two-way valve (64).

36. The ion selective electrode analyzer (1) of claim 35, wherein if the first valve is a pinch valve (61), the ion selective electrode analyzer (1) further comprises a Y-shape connector (62).

37. The ion selective electrode analyzer (1) of claim 35, wherein the ion selective electrode analyzer (1) further comprises a second reagent supply line (11).

38. (canceled)

39. (canceled)

40. The ion selective electrode analyzer (1) of claim 35, wherein at least one of the first pump (80) or second pump (81) is connected to the drain line (50) via the second valve (63), wherein the second valve (63) is positioned at or near the middle of the drain line (50).

41. The ion selective electrode analyzer (1) of claim 35, wherein the third valve (64) is positioned at or near the middle of the flushing liquid line (70) connecting the second pump (81) and the third pump (82).

42. The ion selective electrode analyzer (1) of claim 35, wherein the ion selective electrode analyzer (1) further comprises a fourth pump (83) wherein the fourth pump (83) is connected to the second reagent supply line (11).

43. (canceled)

44. The ion selective electrode analyzer (1) of claim 35, wherein the ion electrode analyzer (1) further comprises a drain (52), wherein the drain line (50) is operatively connected to the drain (52).

45-48. (canceled)
